(19) United States
(12) Patent Application Publication
(10) Pub. No.: US 2017/0000079 A1
(43) Pub. Date: Jan. 5, 2017

(54) ANIMAL LITTER AND METHOD OF PRODUCING SAME

(71) Applicant: WING KEUNG LAU, Tuen Mun (HK)

(72) Inventor: WING KEUNG LAU, Tuen Mun (HK)

(21) Appl. No.: 15/196,285

(22) Filed: Jun. 29, 2016

(30) Foreign Application Priority Data
Jul. 1, 2015 (CN) 201510373907.7

(51) Int. Cl. A01K 1/015; A61L 2/23

(52) U.S. Cl. CPC A01K 1/0155; A01K 1/0154; A61L 2/23

(57) ABSTRACT

The present invention relates to a clumping animal litter, particularly a clumping cat litter, comprising a wood-based liquid-absorptive deodorizing agent, a starch-based adhesion agent capable of adhering to the water-absorptive deodorizing agent, a disintegrant and a clumping agent to clump the litters together when they are in contact with the liquid. The cat litter has high deodorizing and absorptive capability, and which is made of natural and environment-friendly materials. In addition, the present invention aims to provide a method for producing such an animal litter.

### TECHNICAL FIELD OF THE INVENTION

This invention relates generally to an animal litter, and particularly, to a cat litter that is capable of forming clumps upon absorption of animal urine and/or liquid or moisture from fecal matter. This invention also relates to a method of producing the animal litter.

### BACKGROUND OF THE INVENTION

Many families raise pet animals like cats. Cats have the habitual nature of covering their urine and fecal matter with sands. Making use of this habitual nature of cats, cat litter is used for the treatment of cat urine and fecal matter. Cat litter is usually absorbent and deodorizing, helping to maintain a hygienic home environment. Early-stage cat litter was non-clumping, the purpose of which was simply to cover up the cat urine and fecal matter. Later on, clumping cat litter became available, which is capable of self-clumping upon absorption of cat urine and/or liquid or moisture from fecal matter.

For example, bentonite has been used as cat litter, and provides the advantages of low costs and clumping upon absorption. However, bentonite has the problems of producing a large amount of sawdust and poor absorptive capability. Crystal cat litter has also been developed, which has the advantage of producing no sawdust at all. However, crystal cat litter is easily damped, cannot clump upon absorption of water, and is not deodorizing.

More recently, absorptive polymer resins have been added into cat litter in order to improve the absorptive capability thereof. However, it is difficult to evenly mix the polymer resins with other components of the cat litter. In addition, most polymer resins that are highly absorptive of water are poorly absorptive of salty solutions including urine and liquid from fecal matter.

Furthermore, chemical deodorizing and preservative agents have been used in cat litter. However, these chemicals are harmful to both human health and the environment.

Therefore, there is a need for a self-clumping cat litter that is effectively deodorizing, highly absorptive, and environment-friendly.

### SUMMARY OF THE INVENTION

The present invention has a principle object of providing an animal litter, particularly a cat litter, that is capable of self-clumping upon absorption of animal urine and/or liquid or moisture from fecal matter, capable of eliminating undesired smells, capable of absorbing a large amount of urine and/or liquid or moisture from fecal matter, and preferably made of natural materials such that the cat litter brings no harm to human health or the environment. In addition, the present invention aims to provide a method for producing such an animal litter.

These and other objects are satisfied by the present invention, which, in a first aspect thereof, provides an animal litter, preferably a cat litter, comprising a wood-based liquid-absorptive deodorizing agent, a starch-based adhesion agent for tackifying the water-absorptive deodorizing agent, a disintegrant and a clumping agent to clump the litter together when they are in contact with the liquid.

In one embodiment of the present invention, the liquid-absorptive deodorizing agent is pine sawdust or fir sawdust, preferably the wood sawdust is 60-70 wt % by weight of the animal litter.

In one embodiment of the present invention, the adhesion agent is selected from a group comprising cornmeal, corn flour, wheat meal, wheat flour, cassava starch, white rice meal, white rice flour, glutinous rice meal, glutinous rice flour, and any combination thereof, preferably the adhesion agent is 20-30 wt % by weight of the animal litter.

In one embodiment of the present invention, the disintegrant is sodium carboxymethyl starch, preferably the disintegrant is 1-3 wt %, more preferably 2 wt %, by weight of the animal litter. In another embodiment of the invention, sodium carboxymethyl starch may be replaced by a superabsorbent polymer, for example sodium polyacrylatem.

In one embodiment of the present invention, the clumping agent is selected from a group comprising xanthan gum, carrageenan, sodium alginate, and any combination thereof, preferably the clumping agent is 1-2 wt %, more preferably 1.5 wt %, by weight of the animal litter.

In one embodiment of the present invention, the animal litter further comprises a lubricant and a moisturizing agent for moisturizing the clumps. The lubricant may be a vegetable fat selected from a group comprising soybean oil, peanut oil, rapeseed oil, sesame oil, corn oil, and any combination thereof, and the moisturizing agent may be sodium bicarbonate. Preferably, the lubricant is 1-2 wt %, more preferably 1.5 wt %, by weight of the animal litter, and the moisturizing agent is 0.5-1.5 wt %, more preferably 0.5-1 wt %, by weight of the animal litter.

In one embodiment of the present invention, the animal litter further comprises a preservative agent which is 0.5-1.5% wt %, preferably 1 wt %, by weight of the animal litter. The preservative agent may be selected from a group comprising potassium sorbate, calcium propionate, sodium benzoate, sodium erythorbate, sodium formaldehyde sulfoxylate, and any combination thereof.

In one embodiment of the present invention, the animal litter further comprises an antistatic agent for preventing the litter from attaching to hair or fur of the animal. The antistatic agent may be about 0.05-0.15 wt %, preferably 0.1 wt %, by weight of the animal litter. The antistatic agent may comprise sunflower seed oil and a further vegetable oil selected from the group consisting of corn oil, coconut oil, peanut oil, olive oil, sesame oil and any combination thereof. Preferably, the antistatic agent comprises about 85-95 wt %, preferably 90 wt % of sunflower seed oil and 5%-15%, preferably 10% the further vegetable oil by weight of the antistatic agent.

In one embodiment of the present invention, the animal litter is formulated as cylindrical particles with a length of 6-12 millimeters and a diameter of 3-6 millimeters.

A second aspect of the present invention provides a method of manufacturing an animal litter, comprising:

(1) mixing a wood-based liquid-absorptive deodorizing agent with a starch-based adhesion agent for tackifying the liquid-absorptive deodorizing agent to form a mixture at room temperature;
(2) adding water into the mixture under stirring for 20-30 minutes;
(3) adding a lubricant, a disintegrant, a clumping agent and a moisturizing agent in order into the mixture;
(4) stirring the mixture for 20-30 minutes;
(5) granulating the stirred mixture under sufficient pressure and temperature into litter particles; and
(6) subjecting the litter particles to a drying treatment for 10-15 minutes.

In one embodiment of the present invention, the liquid-absorptive agent deodorizing agent is wood dust; the adhesion agent is selected from a group comprising cornmeal, corn flour, wheat meal, wheat flour, cassava starch, white rice meal, white rice flour, glutinous rice meal, glutinous rice flour, and any combination thereof; the lubricant is a vegetable fat selected from a group comprising soybean oil, peanut oil, rapeseed oil, sesame oil, corn oil, and any combination thereof; the disintegrant is sodium carboxymethyl starch; the clumping agent is selected from a group comprising xanthan gum, carrageenan, sodium alginate, and any combination thereof; and the moisturizing agent is sodium bicarbonate.

In one embodiment of the present invention, by weight of the animal litter, liquid-absorptive agent deodorizing agent is 60-70 wt %, the adhesion agent is 20-30 wt %, the lubricant is 1-2 wt %, the disintegrant is 1-3 wt %, the clumping agent is 1-2 wt %, and the moisturizing agent is 0.5-1.5 wt %.

In one embodiment of the present invention, the granulating step is performed under a pressure of 5 tons and at a temperature of 70-80° C. .

In one embodiment of the present invention, step (3) is followed by adding 0.5-1.5 wt % a preservative agent by weight of the animal litter and an antistatic agent.

### DETAILED DESCRIPTION OF THE INVENTION

The cat litter of the present invention makes use of the high absorptive capability and the natural scent of wood sawdust to degrade and eliminate the odor of cat urine and fecal matter, without the use of harmful chemicals. All the components of the cat litter of the present invention are non-toxic, odor-free, pollution-free and non-corrosive, and they originate from readily accessible natural sources. The major component, wood dust, is a cheap or even discarded material, and can be readily found. In the cat litter of the present invention, the wood sawdust may include sawdust produced from various kinds of trees, for example pine trees and Chinese fir trees, and mainly serves as a liquid-absorptive deodorizing agent. Preferably, the percentage by weight of wood sawdust in the cat litter of the present invention is 60-70%.

The cat litter of the present invention also comprises a starch-based adhesion agent, for example corn powder. The corn powder can also lower the density of the cat litter mainly composed of wood dust, causing to quickly decompose and tackify the particles of the cat litter together upon absorption of liquids. Preferably, the percentage by weight of corn powder in the cat litter of the present invention is 20-30%. The corn powder may be substituted by another starch-based adhesion agent for tackifying the water-absorptive deodorizing agent, such as cornmeal, corn flour, wheat meal, wheat flour, cassava starch, white rice meal, white rice flour, glutinous rice meal, glutinous rice flour, or any combination thereof.

Another essential component of the cat litter of the present invention is sodium carboxymethyl starch, which serves as a disintegrant. Sodium carboxymethyl starch is a starch ether derivative derived from carboxymethyl starch. It is soluble in water under room temperature and forms a colorless, transparent and viscous liquid with emulsifying dispersivity and solid dispersivity, but free from rot and toxicity. It is also used in toothpaste, printing and dyeing, tobacco and pharmaceutical industries as additive to play the role of thickening, stabilizing, emulsifying, moisturizing, bonding and modifying, and significantly improves product quality, taste and flavor. It is safe, nontoxic and free from any side effect. Addition of sodium carboxymethyl starch into the cat litter of the present invention allows the wood sawdust to swell and disintegrate rapidly after in contact with cat urine and/or liquid or moisture from fecal matter, while allowing the continuous permeation of the liquid into the wood sawdust. Upon the disintegration of the particles of the cat litter, the wood sawdust therein emulsifies into a non-toxic and harmless denaturated starch, which has the tendency of clumping together. Preferably, the percentage by weight of sodium carboxymethyl starch in the cat litter of the present invention is 1-3%, preferably around 2%.

As an alternative, sodium carboxymethyl starch may be replaced by a superabsorbent polymer, for example sodium polyacrylatem. The superabsorbent polymers are liquid-absorbing polymers that can absorb and retain extremely large amounts of a liquid.

The cat litter of the present invention further comprises a clumping agent that assists to clump the litters together. The clumping agent is preferably xanthan gum, but can also be carrageenan, sodium alginate, or any other suitable clumping agent known in the art that is able to clump the litter together when they are in contact with liquid. Xanthan gum is a polysaccharide secreted by the bacterium Xanthomonas campestris, used as a food additive and rheology modifier, commonly used as a food thickening agent and a stabilizer. It is composed of pentasaccharide repeat units, comprising glucose, mannose, and glucuronic acid in the molar ratio 2:2:1. One of the most remarkable properties of xanthan gum is its ability to produce a large increase in the viscosity of a liquid by adding a very small quantity of gum, on the order of one percent, which property is utilized in the cat litter of the present invention to facilitate the clumping of the cat litter upon absorption of liquids. Preferably, the percentage by weight of xanthan gum in the cat litter of the present invention is 1-2%, preferably around 1.5%.

The above-described essential components present in the cat litter of the present invention provide the advantage that when in contact with cat urine and/or liquid or moisture from fecal matter, the cat litter can absorb the liquid rapidly, and at the same time swell and disintegrate, allowing the odor of the cat urine and/or liquid moisture from fecal matter to be absorbed into the interior of the cat litter. In addition, the degenerated wood sawdust coagulates instantly, fowling clumps which can be easily removed from the rest of the cat litter, for example with a scoop. Thus, the cat urine and/or liquid or moisture from fecal matter can be timely removed, while keeping the remaining un-clumped cat litter unaffected, improving the utilization of the cat litter.

As the cat litter of the present invention is produced mainly from wood sawdust instead of mineral materials (for example, clay including kaolin and bentonite, coal powder, slag, active charcoal, etc.) or chemicals commonly used in the art for the production of cat litter, the production costs of the cat litter are significantly reduced, and the production process is simplified, while avoiding the potential harms from the chemicals contained in the mineral materials.

In addition, wood sawdust has a relatively low density, high porosity and high absorptive capability. Therefore, and cat litter comprising the wood sawdust according to the invention is low in density and has a high porosity. In other words, both the surface and the interior of the particles of the cat litter are alveolate, which is especially beneficial for the absorption and storage of cat urine and or liquid or moisture from fecal matter.

All of the essential components of the cat litter of the present invention (that is, the wood dust, the corn powder, the sodium carboxymethyl starch, and the xanthan gum) are non-toxic, odor-free and degradable, resulting in a non-polluting and environment-friendly cat litter.

Besides the above-described essential components, additional components may also be added into the cat litter of the present invention to render or improve desirable properties. For example, vegetable fat may be added into the cat litter as a lubricant for lubricating the litter particles, at a percentage by weight of 1-2%, preferably around 1.5%. The addition of the lubricant may prevent the clogging caused by the particles in the manufacturing process. The vegetable fat may be selected from a group comprising soybean oil, peanut oil, rapeseed oil, sesame oil, corn oil, and any combination thereof. In addition to improving viscosity, the vegetable fat can also make the surfaces of the particles of the cat litter smooth and preventing jamming of the machines during the production thereof.

A moisturizing agent such as sodium bicarbonate may also be added into the cat litter of the present invention at a percentage by weight of 0.5-1.5%, preferably 0.5-1%. As sodium bicarbonate is easily decomposed at room temperature, the use of sodium bicarbonate can help to moisturize the cat litter, preventing the clumped cat litter from cracking or bursting, and can help to degrade the amino acid in the cat urine.

Furthermore, the cat litter of the present invention may further comprise a preservative agent, the percentage of which is 0.5-1.5%, preferably around 1% by weight. The preservative agent may be selected from a group comprising potassium sorbate, calcium propionate, sodium benzoate, sodium erythorbate, sodium formaldehyde sulfoxylate, and any combination thereof.

One of the distinctive feature of the invention is that the animal litter further comprises about 0.1 wt % of an antistatic agent for preventing the litter from attaching to hair or fur of the animal. Advantageously, the antistatic agent consists of about 90% of sunflower seed oil and about 10% of corn oil. It has been found that this antistatic agent can prevent the litters from attaching to the hair or fur of the animal, thereby maintaining the hygiene of the animal.

The cat litter of the present invention is preferably formulated as cylindrical particles with lengths of 6-12 millimeters and diameters of 3-6 millimeters, the density of which being 0.5-0.8kg/cm³, and the porosity of which being 40-50%. However, those skilled in the art would understand that other shapes and sizes are also possible.

A second aspect of the present invention provides a method of producing an animal litter, in particular a cat litter. In methods of producing animal litter in the prior art, the various components of the cat litter are usually dissolved into a mixture solution. In contrast, the method of the present invention mixes the various components of the cat litter in a relatively dry condition, and forms the particles of the cat litter directly from the solid state components, which reduce the production costs and simplify the production process.

In particular, the method of the present invention begins with mixing a wood-based liquid-absorptive deodorizing agent with a starch-based adhesion agent for tackifying the liquid-absorptive deodorizing agent to form a mixture at room temperature. In this embodiment, the liquid-absorptive deodorizing agent is wood dust, and the percentage by weight thereof in the manufactured cat litter is 60-70 wt %. The starch-based adhesion agent can be selected from a group comprising cornmeal, corn flour, wheat meal, wheat flour, cassava starch, white rice meal, white rice flour, glutinous rice meal, glutinous rice flour, and any combination thereof. In this embodiment, the adhesion agent is cornmeal, and the percentage by weight thereof in the manufactured cat litter is 20-30 wt %. Then, a suitable amount (e.g. 3%) of water is added to the mixture under stirring for 20-30 minutes. After that, optionally, a lubricant such as a vegetable fat can be added to the mixture, at a percentage by weight of 1-2%, preferably 1.5%. The vegetable fat can be selected from a group comprising soybean oil, peanut oil, rapeseed oil, sesame oil, corn oil, and any combination thereof. Then, a disintegrant, a clumping agent, and optionally a moisturizing agent such as sodium bicarbonate and a preservative agent, are sequentially added to the mixture.

In one preferred embodiment, the disintegrant is sodium carboxymethyl starch, and the percentage by weight thereof in the manufactured cat litter is 1-3%, preferably 2%; the clumping agent can be selected from a group comprising xanthan gum, carrageenan, sodium alginate, and any combination thereof, but is preferably xanthan gum, and the percentage by weight thereof in the manufactured cat litter is 1-2%, preferably 1.5%; the percentage by weight of sodium bicarbonate in the manufactured cat litter is 0.5-1.5%, preferably 0.5-1%; the preservative agent can be selected from a group comprising potassium sorbate, calcium propionate, sodium benzoate, sodium erythorbate, sodium formaldehyde sulfoxylate, and any combination thereof, and the percentage by weight thereof in the manufactured cat litter is 0.5-1.5%, preferably 1%. Then the mixture is stirred for another 20-30 minutes. After that, the mixture is transferred into a granulating machine, where the mixture is processed under a pressure of 5 tons and at a temperature of 70-80° C. to foam cylindrical particles with lengths of 6-12 millimeters and diameters of 3-6 millimeters. Finally, the particles are dried with a heat flow drying machine for 10-15 minutes.

Several examples of the process of producing the cat litter of the present invention are provided as follows.

### EXAMPLE 1

A cat litter is produced by the following steps:

(1) mixing 63.5 wt % pine sawdust with 30 wt % corn powder to form a mixture at room temperature;
(2) adding a suitable amount (e.g. 3 wt %) of water to the mixture under stirring for 20-30 minutes;
(3) adding around 1.5 wt % soybean oil, around 2 wt % sodium carboxymethyl starch, around 1.5 wt % xanthan gum, around 0.5 wt % sodium bicarbonate, around 1 wt % potassium sorbate in order into the mixture;
(4) stirring the mixture for 20-30 minutes;
(5) granulating the mixture into cylindrical particles with lengths of 6-12 millimeters and diameters of 3-6 millimeters, under a pressure of 5 tons and at a temperature of 70° C.;
(6) drying the particles with a heat flow drying machine for 10-15 minutes.

### EXAMPLE 2

A cat litter is produced by the following steps:

(1) mixing 65 wt % pine sawdust with 28.2 wt % wheat flour to form a mixture at room temperature;
(2) adding a suitable amount (e.g. 3 wt %) of water to the mixture under stirring for 20-30 minutes;
(3) adding around 1.5 wt % peanut oil, around 2 wt % sodium carboxymethyl starch, around 1.5 wt % carrageenan, 0.8 wt % sodium bicarbonate, 0.5 wt % calcium propionate, 0.5 wt % sodium benzoate in order into the mixture;
(4) stirring the mixture for 20-30 minutes;
(5) granulating the mixture into cylindrical particles with lengths of 6-12 millimeters and diameters of 3-6 millimeters, under a pressure of 5 tons and at a temperature of 75° C.;
(6) drying the particles with a heat flow drying machine for 10-15 minutes.

### EXAMPLE 3

A cat litter is produced by the following steps:

(1) mixing 70 wt % pine sawdust with 23 wt % cassava starch to form a mixture at room temperature;
(2) adding a suitable amount (e.g. 3 wt %) of water to the mixture under stirring for 20-30 minutes;
(3) adding around 1.5 wt % sesame oil to the mixture, around 2 wt % sodium carboxymethyl starch, around 1.5 wt % sodium alginate, 1 wt % sodium bicarbonate, 1 wt % sodium erythorbate, 0.1 wt % a mixture of sunflower seed flower and corn oil (9:1) in order into the mixture;
(4) stirring the mixture for 20-30 minutes;
(5) granulating the mixture into cylindrical particles with lengths of 6-12 millimeters and diameters of 3-6 millimeters, under a pressure of 5 tons and at a temperature of 80° C.;
(6) drying the particles with a heat flow drying machine for 10-15 minutes.

While the present invention is described in connection with what is presently considered to be the most practical and preferred embodiment, it should be appreciated that the invention is not limited to the disclosed embodiment, and is intended to cover various modifications and equivalent arrangements included within the scope of the claims. Modifications and variations in the present invention may be made without departing from the novel aspects of the invention as defined in the claims, and this application is limited only by the scope of the claims.

What is claimed is:

1. An animal litter, comprising a wood-based liquid-absorptive deodorizing agent, a starch-based adhesion agent for tackifying the water-absorptive deodorizing agent, a disintegrant and a clumping agent to clump the litters together when they are in contact with the liquid.

2. The animal litter of claim 1, wherein the liquid-absorptive deodorizing agent is pine sawdust or fir sawdust, and the sawdust is 60-70 wt % by weight of the animal litter.

3. The animal litter of claim 1, wherein the adhesion agent is selected from a group comprising cornmeal, corn flour, wheat meal, wheat flour, cassava starch, white rice meal, white rice flour, glutinous rice meal, glutinous rice flour, and any combination thereof, and the adhesion agent is 20-30 wt % by weight of the animal litter.

4. The animal litter of claim 1, wherein the disintegrant is sodium carboxymethyl starch, and the disintegrant is 1-3 wt %, preferably 2 wt %, by weight of the animal litter.

5. The animal litter of claim 4, wherein sodium carboxymethyl starch is replaced by a superabsorbent polymer, preferably sodium polyacrylatem.

6. The animal litter of claim 1, wherein the clumping agent is selected from a group comprising xanthan gum, carrageenan, sodium alginate, and any combination thereof, and the clumping agent is 1-2 wt %, preferably 1.5 wt %, by weight of the animal litter.

7. The animal litter of claim 1, further comprising a lubricant and a moisturizing agent for moisturizing the clumps.

8. The animal litter of claim 7, wherein the lubricant is a vegetable fat selected from a group comprising soybean oil, peanut oil, rapeseed oil, sesame oil, corn oil, and any combination thereof, and the moisturizing agent is sodium bicarbonate.

9. The animal litter of claim 7, wherein the lubricant is 1-2 wt %, preferably 1.5 wt %, by weight of the animal litter, and the moisturizing agent is 0.5-1.5 wt %, preferably 0.5-1 wt %, by weight of the animal litter.

10. The animal litter of claim 1, further comprising a preservative agent which is 0.5-1.5% wt %, preferably 1 wt %, by weight of the animal litter.

11. The animal litter of claim 10, wherein the preservative agent is selected from a group comprising potassium sorbate, calcium propionate, sodium benzoate, sodium erythorbate, sodium formaldehyde sulfoxylate, and any combination thereof.

12. The animal litter of claim 1, further comprising an antistatic agent for preventing the litter from attaching to hair or fur of the animal.

13. The animal litter of claim 12, wherein the antistatic agent comprises sunflower seed oil and a further vegetable oil selected from the group consisting of corn oil, coconut oil, peanut oil, olive oil, sesame oil and any combination thereof.

14. The animal litter of claim 1, wherein the antistatic agent comprises about 85-95 wt %, preferably 90 wt % of sunflower seed oil and 5%-15%, preferably 10% the further vegetable oil by weight of the antistatic agent.

15. The animal litter of claim 1, formulated as cylindrical particles with a length of 6-12 millimeters and a diameter of 3-6 millimeters.

16. A method of manufacturing an animal litter, comprising:
(1) mixing a wood-based liquid-absorptive deodorizing agent with a starch-based adhesion agent capable of adhering to the liquid-absorptive deodorizing agent to form a mixture at room temperature;
(2) adding water into the mixture under stirring for 20-30 minutes;
(3) adding a lubricant, a disintegrant, a clumping agent and a moisturizing agent in order into the mixture;
(4) stirring the mixture for 20-30 minutes;
(5) granulating the stirred mixture under sufficient pressure and temperature into litter particles; and
(6) subjecting the litter particles to a drying treatment for 10-15 minutes.

17. The method of claim 16, wherein the liquid-absorptive agent deodorizing agent is wood dust; the adhesion agent is selected from a group comprising cornmeal, corn flour, wheat meal, wheat flour, cassava starch, white rice meal, white rice flour, glutinous rice meal, glutinous rice flour, and any combination thereof; the lubricant is a vegetable fat selected from a group comprising soybean oil, peanut oil, rapeseed oil, sesame oil, corn oil, and any combination thereof; the disintegrant is sodium carboxymethyl starch; the clumping agent is selected from a group comprising xanthan gum, carrageenan, sodium alginate, and any combination thereof; and the moisturizing agent is sodium bicarbonate.

18. The method of claim 17, wherein by weight of the animal litter, liquid-absorptive agent deodorizing agent is 60-70 wt %, the adhesion agent is 20-30 wt %, the lubricant is 1-2 wt %, the disintegrant is 1-3 wt %, the clumping agent is 1-2 wt %, and the moisturizing agent is 0.5-1.5 wt %.

19. The method of claim 16, wherein the granulating step is performed under a pressure of 5 tons and at a temperature of 70-80° C.

20. The method of claim 16, wherein step (3) is followed by adding 0.5-1.5 wt % a preservative agent by weight of the animal litter and an antistatic agent.
